(19) United States
(12) Patent Application Publication
(10) Pub. No.: US 2022/0401038 A1
(43) Pub. Date: Dec. 22, 2022

(54) METHOD FOR PREDICTING DECOMPENSATION EVENTS IN NASH PATIENTS

(71) Applicant: MERIDIAN BIOSCIENCE ISRAEL LTD, Modi'in (IL)

(72) Inventors: Gil GUGGENHEIM, Jerusalem (IL); Ofer SCHLESINGER, Rehovot (IL); Avraham HERSHKOWITZ, Nof-Ayalon (IL); Ilan BEN-OREN, Modiin (IL); Raphael WERNER, Kohav-Yair (IL)

(21) Appl. No.: 17/777,362

(22) PCT Filed: Nov. 16, 2020
(86) PCT No.: PCT/IL2020/051183

§ 371 Date: May 17, 2022

Related U.S. Application Data
(60) Provisional application No. 62/937,486, filed on Nov. 19, 2019.

(51) Int. Cl. A61B 5/00; A61B 5/08

(52) U.S. Cl. CPC A61B 5/7275; A61B 5/082; A61B 5/4244; A61B 5/4842

(57) ABSTRACT

Disclosed is a method for predicting a decompensation event in a patient suffering from NASH, including performing a ¹³C methacetin breath test on a patient; calculating a PDR-peak value of the patient, based on the ¹³C methacetin breath test; and identifying the patient as having high-risk of developing a decompensation event within 1 year from the performing of the breath test, if the PDR-peak value is at or below a predetermined threshold value; wherein the predetermined threshold value is 15%/h.

### FIELD OF TECHNOLOGY

The present application generally relates to the field of non-invasive monitoring and prioritizing of patients suffering from NASH, in particular patients suffering from decompensated cirrhotic NASH based on a methacetin breath test.

### BACKGROUND

Liver disease has become one of the most common chronic illnesses, affecting tens of millions of people in the developed world, resulting in lifetime suffering and huge costs to the medical system. Nonalcoholic fatty liver disease (NAFLD) has become a common chronic liver condition due to obesity and diabetes mellitus, affecting almost a quarter of the general population in the United States. The trend of a rise in obesity in the western world is increasing annually. NAFLD includes a spectrum of liver conditions, ranging from simple steatosis (fat accumulation), also referred to as nonalcoholic fatty liver (NAFL), to non-alcoholic steatohepatitis (NASH) disease, which is associated with liver injury. NASH may progress from fibrosis to cirrhosis, as a consequence of the distortion of the normal liver architecture that interferes with blood flow through the liver. Cirrhosis can also lead to an inability of the liver to perform its biochemical functions, resulting in complications that cause liver failure and liver cancer. Transplantation then becomes the only feasible solution, but in many cases, even transplantation is not an option. Currently, there are several treatments in the pipeline for NASH, but there is no known approved and effective treatment currently available.

Breath tests that are based on monitoring the ¹³CO₂, which is a by-product of metabolization by the liver of ¹³C labeled substrates, have been proposed as a tool for evaluation of liver function.

Methacetin may be utilized for the evaluation of liver functional capacity and/or the extent of liver injury. The biochemical basis for the evaluation of functional capacity is that the compound is metabolized by a cytochrome p450 enzyme expressed in normal liver cells (hepatocytes).

One of the most common methods for determining the rate of metabolism of methacetin is to analyze the rate of metabolism of the methoxy group (CH3O) of methacetin to carbon dioxide, which is excreted in exhaled breath. To distinguish the carbon dioxide derived from methacetin from all other sources of carbon dioxide, the methoxy group is labeled with ¹³C, a stable isotope of carbon. Thus, all the CO₂ derived from methacetin will contain ¹³C (¹³CO₂) in contrast to all other sources of CO₂, which will contain approximately 99% ¹²C, and 1% ¹³C, the naturally abundant isotope. Thus, the rate of excretion of ¹³CO₂ (normalized to ¹²CO₂) above background following the administration of methacetin-methoxy-¹³C indicates its rate of metabolism, which relates to the hepatic cell “health” and to the functional mass of the liver.

The Model for End-Stage Liver Disease, or MELD, is a scoring system for assessing the severity of chronic liver disease. The score is now used by the United Network for Organ Sharing (UNOS) and Eurotransplant for prioritizing allocation of liver transplants instead of the older Child-Turcotte-Pugh (CTP, also known as Child-Pugh score or just Child criteria) score, which included subjective components in its score (such as ascites or hepatic encephalopathy grading).

MELD uses the patient's values for serum bilirubin, serum creatinine, and the international normalized ratio for prothrombin time (INR) to predict survival and in more advanced patients also sodium (Na) level. It is calculated according to the following formula.

MELD=3.78×ln[serum bilirubin (mg/dL)]+11.2×ln[INR]+9.57×ln[serum creatinine (mg/dL)]+6.43.

For patients with an initial MELD score greater than 11, the MELD score is then re-calculated as follows:

MELD=MELD(i)+1.32*(137−Na)−[0.033*MELD(i)*(137−Na)]

However, it has been observed that patients evaluated with a same clinical diagnosis and MELD score may have different risk of deterioration and/or future risk of a decompensation event.

There therefore is an unmet need for non-invasive, point-of-care prognostic tool which can help identifying patients having an elevated risk of hepatic decompensation and/or deterioration of liver function.

### SUMMARY OF THE INVENTION

Aspects of the disclosure, in some embodiments thereof, relate to methods for predicting deterioration in a NASH patient's liver function based on a non-invasive methacetin breath test, wherein the deterioration comprises predicting the probability of a decompensation event taking place within one year or less from the taking of the breath test, specifically wherein the deterioration comprises predicting the probability of in the MELD score of the patient to occur within 1 year of the taking of the breath-test. According to some embodiments, the increase is a at least a 2-point increase, at least 3-point increase or at least a 4-point increase in the MELD-score.

As used herein, the term “decompensation event” is defined by the occurrence of at least one of: liver-specific or all-cause mortality, liver transplant, variceal hemorrhage that require ‘non-drug therapy’ (e.g. variceal ligation, sclerotherapy), portal hypertensive gastropathy hemorrhage requiring hospitalization, variceal hemorrhage or occurrence of large gastroesophageal varices or any varices with red wale sign (classified as severe) and that required ‘non-drug therapy’ (e.g. variceal ligation or sclerotherapy), ascites requiring diuretics (in a subject without prior history of ascites requiring diuretics) or refractory ascites requiring ‘non-drug therapy’ (e.g. paracentesis), new onset or worsening of hepatic encephalopathy (HE) requiring hospitalization in the absence of known co-morbidities or alternative causes, hepatorenal syndrome requiring hospitalization, spontaneous bacterial peritonitis (SBP) requiring hospitalization, and MELD-score progression defined as any increase ≥4 points from baseline. Each possibility is a separate embodiment.

Advantageously, the method may assist in prioritizing the allocation of liver transplants by identifying patients having higher risk of deterioration and/or a more urgent risk of deterioration. According to some embodiments, the method may enable identifying patients having a risk of developing a decompensation event and optionally require an urgent liver transplantation within a short period of time, e.g. in less than a year, less than half a year or less than three months, optionally even despite them currently having a relatively low MELD-score. According to some embodiments, the method may assist in determining “per-month accumulated” risk assessment, such as the risk of the patient developing a decompensation event and/or having an at least 2-point, at least a 3-point or at least a 4-point increase in his/her MELD score the urgency within 1-month from the breath test, within 2 months from the breath test, within 3 months from the breath test etc. Each possibility is a separate embodiment.

Advantageously, the method may also assist in determining which patients require more close monitoring

According to some embodiments, the method comprises predicting at least a MELD-score progression defined as any increase ≥4 points from baseline.

According to some embodiments, there is provided a method for predicting progression in a MELD-score of a patient suffering from NASH, the method comprising: performing a ¹³C methacetin breath test on a patient; the patient suffering from NASH and having a baseline MELD-score; calculating a PDR-peak value of the patient, based on the ¹³C methacetin breath test; and identifying the patient as having high-risk of MELD-score progression, if the PDR-peak value is at or below a predetermined threshold value, wherein progression of the MELD score comprises an increase of at least 4 points from the baseline MELD-score within 1 year from the performing of the breath test and wherein the predetermined threshold value is about 15%/h, about 10%/h, about 7.5%/h or about 5.5%/h. Each possibility is a separate embodiment.

As used herein, the term “increased risk”, “higher risk”, “increased probability” and “higher probability” may be used interchangeably and refer to the patients being at least twice or at least three times as likely of deterioration (developing a decompensation event, experiencing an at least a 2-point, at least a 3-point or at least 4-point MELD-score increase and/or requiring a liver transplantation) than patient who are not identified as being high-risk. Each possibility is a separate embodiment.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein the term “similar”, with referral to a MELD-score refers to an identical MELD-score or a MELD-score deviating by less than 4 points, less than 3 points or less than 2 points. Each possibility is a separate embodiment.

According to some embodiments, the method further comprises intensifying monitoring of the patient if the patient is identified as having high risk of MELD-score progression as compared to patients identified with a lower risk of MELD-score progression.

Additionally or alternatively, the method further comprises promoting the priority of the patient on a transplant priority list, if the patient is identified as having high risk of MELD-score progression vis-à-vis patients identified with a lower risk of MELD-score progression, optionally despite the other patient having a same or similar base-line MELD-score (e.g. ±2 points) and optionally even despite the other patient having a higher MELD-score than the patient identified as having a high risk of MELD-score progression.

Additionally, or alternatively, the method further comprises changing/adjusting the management of the patient, if the patient is identified as having high risk of MELD-score progression. According to some embodiments, the changing/adjusting comprises prescribing drugs such as antibiotics to avoid infections or statins to reduce inflammation or non-selective beta-blockers to reduce portal hypertension, avoiding certain interventions such as surgeries, changes in diet, and the like. Each possibility is a separate embodiment.

According to some embodiments, the method further comprises predicting a decompensation event in the patient if the PDR-peak value is at or below of the predetermined threshold. According to some embodiments, the decompensation event comprises occurrence of at least one of: hepatic encephalopathy, ascites, variceal hemorrhage, portal hypertensive gastropathy hemorrhage, spontaneous bacterial peritonitis, hepatorenal Syndrome, death or any combination thereof.

According to some embodiments, the patient having a PDR-peak value at or below the predetermined threshold value is at least 3 times more likely to develop a decompensation event or MELD progression than patients having a PDR-peak value above the predetermined threshold value.

According to some embodiments, the patient is suffering from cirrhotic NASH. According to some embodiments, the patient is suffering from decompensated cirrhotic NASH.

As used herein the term “compensated NASH” refer to NASH patients who have not yet experienced a decompensation event. As used herein the term “decompensated NASH” refer to NASH patients who have already had at least one decompensation event. Typically, decompensated NASH patients are patients with cirrhosis that has progressed to the point that the liver is having trouble functioning.

According to some embodiments, the method comprises identifying the patient as having a high-risk of experiencing an at least 2-point, at least 3-point or at least 4-point increase in MELD-score within 6 months of the breath-test if the PDR-peak value is at or below the predetermined threshold value. Each possibility is a separate embodiment.

According to some embodiments, the method further comprises providing a monthly accumulated risk score based on the PDR-peak value.

As used herein, the term “monthly accumulated risk score” refers to a score indicative of the risk of the patient developing a decompensation event and/or at least a 2-point, at least 3-point or at least 4-point increase in MELD-score over time. Each possibility is a separate embodiment.

As a non-limited example, the accumulated score may indicate that the patient has a first risk of developing a decompensation event and/or at least a 4-point increase in MELD-score as soon as within 1-month from the taking of the breath test, and a second risk (higher than the first risk of developing a decompensation event and/or at least a 4-point increase in MELD-score within two months etc., thereby assisting in establishing the urgency of the patient's condition.

According to some embodiments, there is provided a method for predicting a decompensation event in a patient suffering from NASH, the method comprising: performing a ¹³C methacetin breath test on a patient; calculating a PDR-peak value of the patient, based on the ¹³C methacetin breath test; and identifying the patient as having high-risk of developing a decompensation event within 1 year from the performing of the breath test, if the PDR-peak value is at or below a predetermined threshold value; wherein the predetermined threshold values is about 15%/h, about 10%/h, about 7.5%/h or about 5.5%/h. Each possibility is a separate embodiment.

According to some embodiments, the method further comprises intensifying monitoring of the patient if the patient is identified as having high risk of developing a decompensation event, as compared to patients identified with a lower risk of developing a decompensation event.

Additionally or alternatively, the method further comprises promoting the priority of the patient on a transplant priority list, if the patient is identified as having high risk of developing a decompensation event vis-à-vis patients identified with a lower risk of developing a decompensation event, optionally despite the other patient having a same or similar base-line MELD-score (e.g. ±2 points) and optionally even despite the other patient having a higher MELD-score than the patient identified as having a high risk of developing a decompensation event.

Additionally, or alternatively, the method further comprises changing/adjusting the management of the patient, if the patient is identified as having high risk of developing a decompensation event. According to some embodiments, the changing/adjusting comprises prescribing drugs such as antibiotics to avoid infections or statins to reduce inflammation or non-selective beta-blockers to reduce portal hypertension, avoiding certain interventions such as surgeries, changes in diet, and the like. Each possibility is a separate embodiment.

According to some embodiments, the patient having a PDR-peak value at or below the predetermined threshold value is at least 3 times more likely to develop a decompensation event than patients having a PDR-peak value above the predetermined threshold value.

According to some embodiments, the decompensation event comprises occurrence of at least one of: hepatic encephalopathy, ascites, variceal hemorrhage, portal hypertensive gastropathy hemorrhage, spontaneous bacterial peritonitis, hepatorenal Syndrome, at least 2-point, at least 3-point or at least 4-point progression in MELD score, death or any combination thereof.

According to some embodiments, the patient is suffering from cirrhotic NASH. According to some embodiments, the patient is suffering from decompensated cirrhotic NASH.

According to some embodiments, the method comprises identifying the patient as having a high-risk of experiencing an at least 2-point, at least 3-point or at least 4-point increase in MELD-score within 6 months of the breath-test if the PDR-peak value is at or below the predetermined threshold value.

According to some embodiments, the method further comprises providing a monthly accumulated risk score based on the PDR-peak value.

As used herein, the term “monthly accumulated risk score” refers to a score indicative of the risk of the patient developing a decompensation event and/or at least a 2-point, at least 3-point or at least 4-point increase in MELD-score over time. As a non-limited example, the accumulated score may indicate that the patient has a first risk of developing a decompensation event and/or at least a 2-point, at least 3-point or at least 4-point increase in MELD-score as soon as within 1-month from the taking of the breath test, and a second risk (higher than the first risk of developing a decompensation event and/or at least a 2-point, at least 3-point or at least 4-point increase in MELD-score within two months etc., thereby assisting in establishing the urgency of the patient's condition.

According to some embodiments, there is provided a method for prioritizing NASH patients for allocation of liver transplants, the method comprising: obtaining or producing a transplantation priority list for allocation of transplants; obtaining a baseline MELD-score from a patient appearing on or being a candidate to the priority list; the patient suffering from NASH, obtaining ¹³C methacetin breath test results of the patient and calculating a PDR-peak value of the patient, based on the ¹³C methacetin breath test or directly obtaining the PDR-peak value of the patient, and if the calculated PDR-peak value is at or below a predetermined threshold value, promoting the priority of the patient on the transplant priority list relative to patients having a similar MELD-score and a PDR-peak value above the predetermined threshold value; wherein the predetermined threshold values is about 15%/h, about 10%/h, about 7.5%/h or about 5.5%/h. Each possibility is a separate embodiment.

According to some embodiments, the patient is suffering from cirrhotic NASH. According to some embodiments, the patient is suffering from decompensated cirrhotic NASH.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show details of an embodiment in more detail than is necessary for a fundamental understanding of the teachings of the disclosure.

FIG. 1 is a flowchart of a method for predicting progression in a MELD-score of a patient suffering from NASH, according to some embodiments;

FIG. 2 is a flowchart of a method for predicting occurrence of a decompensation event in a patient suffering from NASH, according to some embodiments;

FIG. 3 is a flowchart of a method for prioritizing NASH patients for allocation of liver transplants, according to some embodiments;

FIG. 4 shows results obtained when evaluating the capability of the method to predict occurrence of a decompensation events.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

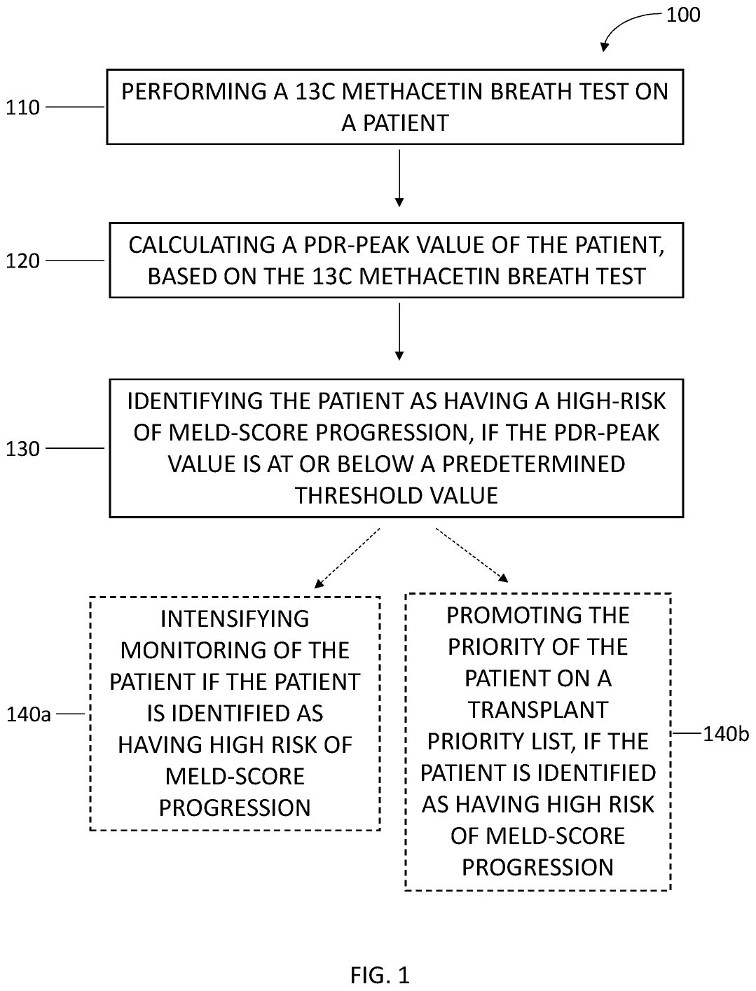

Reference is now made to FIG. 1 which is a flowchart of a method 100 for predicting progression in a MELD-score of a patient suffering from NASH and having a predetermined baseline MELD-score.

In step 110 a ¹³C methacetin breath test is performed on the patient followed by a calculating of at least a PDR-peak value of the patient, based on the ¹³C methacetin breath test (step 120). In step 130 the patient is identified as having high-risk of MELD-score progression, if the PDR-peak value is at or below a predetermined threshold value, wherein progression of the MELD score is defined as an increase of at least 4 points from the predetermined baseline MELD-score within 1 year (or less) from the performing of the breath test. According to some embodiments, the predetermined threshold value is at or below a PDR-peak value in the range of 5%/h-15%/h. The actual threshold value chosen is a question of risk-management and preferences. It is understood that when the threshold is set closer to 15%/h (e.g. 11%/h) the risk of wrongful exclusion of a high-risk patient is diminished however many non-high-risk patients may be included thus reducing the specificity of the method. On the other hand, if a threshold close to 5% is chosen (e.g. 5.5%) the risk of wrongful exclusion of a high-risk patient is increased however the certainty of patients identified as being high-risk actually being high risk significantly improved.

Optionally, method 100 may further include a step 140a of intensifying monitoring of the patient if the patient is identified as having high risk of MELD-score progression, as compared to patients identified with a lower risk of MELD-score progression. Additionally or alternatively, method 100 may further include a step 140b of promoting the priority of the patient on a transplant priority list, if the patient is identified as having high risk of MELD-score progression vis-à-vis patients identified with a lower risk of MELD-score progression, optionally despite the other patient having a same or similar base-line MELD-score (e.g. ±2 points) and optionally even despite the other patient having a higher MELD-score than the patient identified as having a high risk of MELD-score progression. According to some embodiments, the method may further include a step of determining the urgency of the patient's condition, i.e. a risk of MELD-score progression as a function of time, based at least on the PDR-peak value and optionally further on the predetermined baseline MELD score (step not shown).

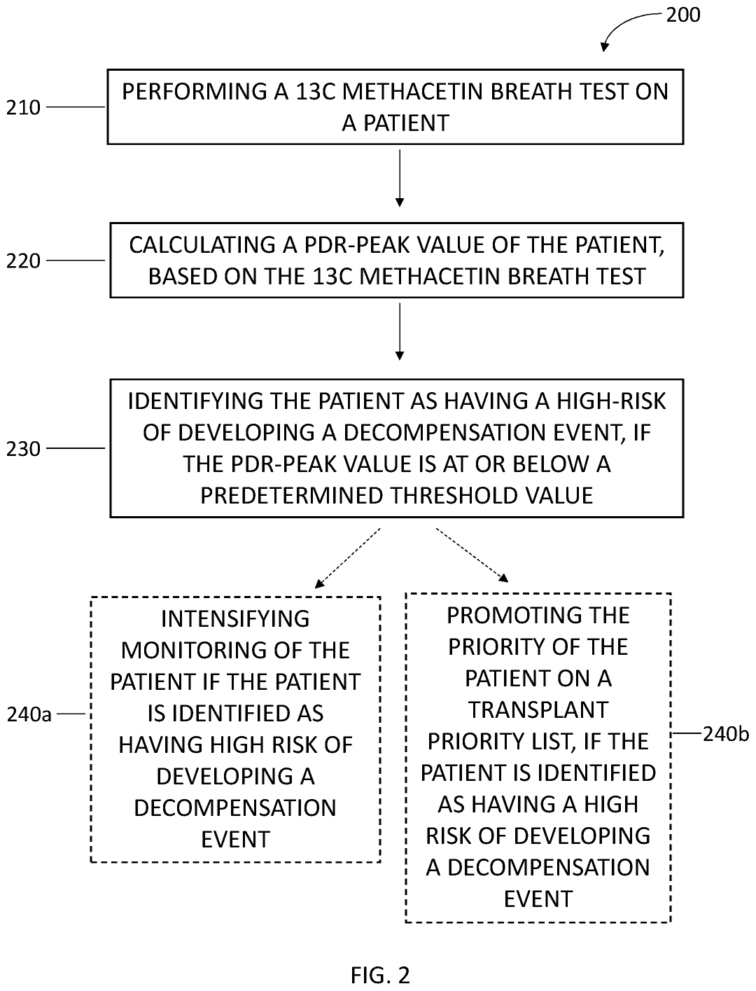

Reference is now made to FIG. 2 which is a flowchart of a method 200 for predicting progression in a MELD-score of a patient suffering from NASH and having a predetermined baseline MELD-score.

In step 210 a ¹³C methacetin breath test is performed on the patient followed by a calculating of at least a PDR-peak value of the patient, based on the ¹³C methacetin breath test (step 220). In step 230 the patient is identified as having high-risk of decompensation event occurrence within 1 year (or less) from the performing of the breath test, if the PDR-peak value is at or below a predetermined threshold value. The decompensation event may be defined as occurrence of at least one of: new onset or worsening of hepatic encephalopathy, new or refractory ascites, variceal hemorrhage, occurrence of large gastroesophageal varices or any varices with red wale sign, portal hypertensive gastropathy hemorrhage, spontaneous bacterial peritonitis, hepatorenal syndrome, at least 2-point, at least 3-point or at least 4-point progression in MELD score, death (liver-specific or all-cause) or any combination thereof.

According to some embodiments, the predetermined threshold value is at or below a PDR-peak value in the range of 5%/h-15%/h. The actual threshold value chosen is a question of risk-management and preferences. It is understood that when the threshold is set closer to 15%/h (e.g. 11%/h) the risk of wrongful exclusion of a high-risk patient is diminished however many non-high-risk patients may be included thus reducing the specificity of the method. On the other hand, if a threshold close to 5% is chosen (e.g. 5.5%) the risk of wrongful exclusion of a high-risk patient is increased however the certainty of patients identified as being high-risk actually being high risk significantly improved.

Optionally, method 200 may further include a step 240a of intensifying monitoring of the patient if the patient is identified as having high risk of developing a decompensation event, as compared to patients identified with a lower risk of developing a decompensation event. Additionally or alternatively, method 200 may further include a step 240b of promoting the priority of the patient on a transplant priority list, if the patient is identified as having high risk of decompensation event occurrence vis-à-vis patients identified with a lower risk of developing a decompensation event, optionally despite the other patient having a same or similar base-line MELD-score (e.g. ±2 points) and optionally even despite the other patient having a higher MELD-score than the patient identified as having a high risk of MELD-score progression.

According to some embodiments, the method may further include a step of determining the urgency of the patient's condition, i.e. a risk of developing a decompensation event as a function of time, based at least on the PDR-peak value and optionally further on the predetermined baseline MELD score of the patient (step not shown).

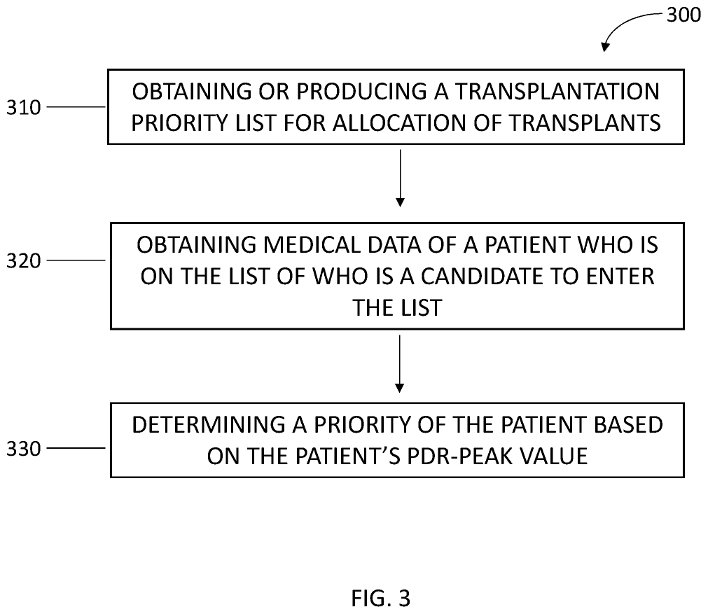

Reference is now made to FIG. 3 which is a flowchart of a method 300 for prioritizing NASH patients for allocation of liver transplants.

In step 310 a transplantation priority list for allocation of transplants is obtained or being generated; in step 320 medical data of a patient who suffers from NASH and who is on the transplantation priority list of who is a candidate to enter the transplantation priority list are obtained. The data include at least a baseline MELD-score and ¹³C methacetin breath test results of the patients. According to some embodiments, the ¹³C methacetin breath test results may be “raw” data in which case step 320 includes calculating a PDR-peak value of the patient, based on the raw ¹³C methacetin breath test data. Alternatively, the PDR-peak value of the patient may be precalculated and directly obtained.

In step 330 a priority of the patient may be determined (number on list) based on the patient's PDR-peak value. That is, if the PDR-peak value is at or below a predetermined threshold value, the patient may receive a higher priority on the transplant priority list relative to patients having a similar baseline MELD-score but a PDR-peak value above the predetermined threshold value.

According to some embodiments, the predetermined threshold value is at or below a PDR-peak value in the range of 5%/h-15%/h. The actual threshold value chosen is a question of risk-management and preferences. It is understood that when the threshold is set closer to 15%/h (e.g. 11%/h) the risk of wrongful exclusion of a high-risk patient (i.e. providing him/her with a lower than “deserved” priority) is diminished; however many non-high-risk patients may be included (thus providing them with a higher than “deserved” priority). On the other hand, if a threshold close to 5% is chosen (e.g. 5.5%) the risk of wrongful exclusion of a high-risk patient is increased; however, the certainty of patients identified as being high-risk actually being high risk significantly improved.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1— Verification of Predictive Capacity of the Method

138 patients suffering from decompensated NASH cirrhosis underwent a ¹³C methacetin breath test using a BreathID® Breath Test Device, every 24 weeks for 1 year.

The medical baseline data of the patients are outlined in Table 1 below

TABLE 1

| | n = 138 | |
|---|---|---|
| Females | 72 | (52.2%) |
| Age (SD) | 61.1 | (8.5) years |
| BMI (SD) | 33.9 | (6.2) kg/m² |
| MELD (SD) | 10.4 | (3.2) |

The below list sets forth events that the prediction clinically evaluated using the herein disclosed method:
All-cause Mortality
MELD score progression defined as any increase ≥4 points from baseline
New onset of Hepatic Encephalopathy
New onset of Ascites
Bleeding Varices

All events were reviewed and adjudicated blinded to the ¹³C methacetin breath test results before efficacy analysis.

Table 2 below outlines the events identified in 38 of the 138 patients evaluated, as determined based on clinical evaluation.

TABLE 2

| Decompensation Event | n = 38 |
|---|---|
| MELD Progression | 25 |
| New onset of Hepatic Encephalopathy | 1 |
| New onset of Ascites | 1 |
| Bleeding Varices | 7 |
| Death | 4 |

A PDR-peak value was calculated for each of the 138 patients and a PDRpeak threshold value was set to 5.5%/h, i.e. patients having a PDRpeak at or below 5.5% were considered to be ‘high risk’ patients.

The ¹³C methacetin breath test results were then tested for their ability to significantly predict event-free survival using the Cox proportional hazards regression model using the above identified threshold value.

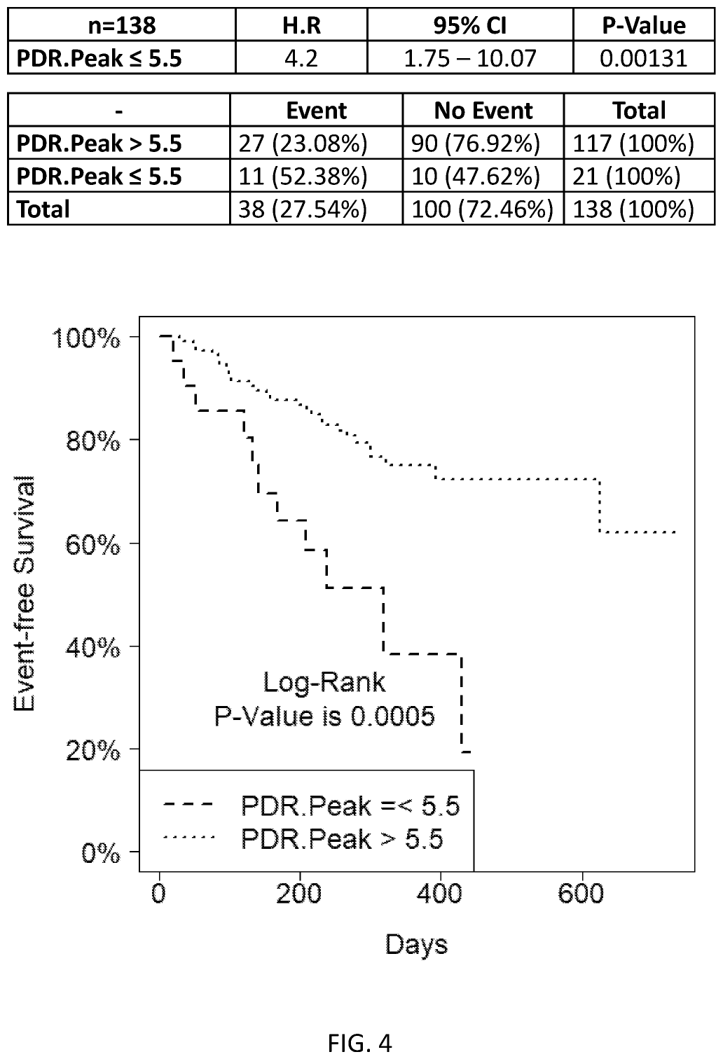

As seen from FIG. 4, the breath test using the 5.5% threshold value provided a hazard ratio (HR) of 4.2 (95% CI: 1.75-10.07 p=0.00131) meaning that patients having a PDR-peak value at or below the threshold were more likely to experience a decompensation event, (in particular an at least 4-point increase in MELD-score (˜66% of the events)). In fact, 52% of the patients ( 11/22) having a PDR-peak value at or below the threshold experienced a decompensation event during the study, as opposed to 23% of the patients having a PDR-peak value above the threshold ( 27/117).

In comparison, the best cut-off using the MELD score was ≥14, resulting in a HR of 2.4 (95% CI: 1.03-5.57 p=0.0414) and the best cut off using the Child-Turcotte-Pugh (CTP) Score was ≥8, resulting in a HR of 2.63 (95% CI: 1.23-5.59 p=0.0123).

In fact, as shown in table 3, multivariate analyses showed that the herein disclosed PDRpeak≤5.5%/h had an independent predictive value superior to currently used predictors such as MELD or CTP scores (whether separately or combined).

TABLE 3
Multivariate Cox Regression with best cut-offs

| Multivariate Cox Regression | HR | 95% Cl | p-value |
|---|---|---|---|
| PDR.Peak ≤ 5.5 | 4.39 | 1.8, 10.71 | 0.00117 |
| MELD ≥ 14 | 2.31 | 0.89, 5.98 | 0.08502 |
| CTP ≥ 8 | 1.79 | 0.77, 4.15 | 0.17402 |

Furthermore, as seen from Table 4 below, the herein disclosed method enabled predicting events occurring half a year and 1 year after the breath test with a similar confidence (HR of 4.72 and 4.27, respectively).

TABLE 4

| | | | |
|---|---|---|---|
| N = 138, | | | |
| PDRₚₑₐₖ ≤ 5.5%/h | HR | 95% CI | P-value |

| | | | | |
|---|---|---|---|---|
| One | Year | 4.27 | 1.72-10.61 | 0.00177 |
| 14 | year | 4.72 | 1.49-14.93 | 0.00836 |

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-26. (canceled)

27. A method for predicting progression in a MELD-score of a patient suffering from NASH, the method comprising:
performing a ¹³C methacetin breath test on a patient; the patient suffering from NASH and having a baseline MELD-score;
calculating a PDR-peak value of the patient, based on the ¹³C methacetin breath test;
identifying the patient as having high-risk of MELD-score progression if the PDR-peak value is at or below a predetermined threshold value, wherein progression of the MELD score comprises an increase of at least 2 points from the baseline MELD-score within 1 year from the performing of the breath test and wherein the predetermined threshold value is 15%/h;
intensifying monitoring of the patient if the patient is identified as having high risk of MELD-score progression as compared to patients identified with a lower risk of MELD-score progression.

28. The method of claim 27, further comprising promoting the priority of the patient on a transplant list, if the patient is identified as having high risk of MELD-score progression vis-à-vis patients identified with a lower risk of MELD-score progression.

29. The method of claim 27, wherein the predetermined threshold value is 5.5%/h.

30. The method of claim 27, further comprising predicting a decompensation event in the patient if the PDR-peak value is at or below of the predetermined threshold value.

31. The method of claim 27, wherein the decompensation event comprises occurrence of at least one of: new onset or worsening of hepatic encephalopathy, new onset or refractory ascites, variceal hemorrhage or occurrence of large gastroesophageal varices or any varices with red wale sign, portal hypertensive gastropathy hemorrhage, spontaneous bacterial peritonitis, hepatorenal syndrome, liver-specific or all-cause death or any combination thereof.

32. The method of claim 27, wherein the patient is suffering from cirrhotic NASH or decompensated cirrhotic NASH.

33. The method of claim 27, wherein the method comprises identifying the patient as having a high-risk of experiencing an at least 2-point increase in MELD-score within 6 months of the breath-test, if the PDR-peak value is at or below the predetermined threshold value.

34. The method of claim 27, further comprising providing a monthly accumulated risk score, based on the PDR-peak value.

35. A method for prioritizing NASH patients for allocation of liver transplants, the method comprising:
obtaining or producing a transplantation priority list for allocation of transplants;
obtaining a baseline MELD-score from a patient appearing on or being a candidate to the transplantation priority list; the patient suffering from NASH obtaining ¹³C methacetin breath test results of the patient;
obtaining or calculating a PDR-peak value of the patient, based on the ¹³C methacetin breath test; and
if the calculated PDR-peak value at or below a predetermined threshold value, promoting the priority of the patient on the transplant priority list, relative to patients having a similar MELD-score and a PDR-peak value above the predetermined threshold value; wherein the predetermined threshold value is 15%/h.

36. The method of claim 35, wherein the predetermined threshold value is 5.5%/h.

37. The method of claim 35, wherein the patient is suffering from cirrhotic NASH or decompensated cirrhotic NASH.

38. A method for predicting a decompensation event in a patient suffering from NASH, the method comprising:
performing a ¹³C methacetin breath test on a patient;
calculating a PDR-peak value of the patient, based on the ¹³C methacetin breath test;
identifying the patient as having high-risk of developing a decompensation event within 1 year from the performing of the breath test, if the PDR-peak value is at or below a predetermined threshold value; wherein the predetermined threshold value is 15%/h; and
intensifying monitoring of the patient if the patient is identified as having high risk of developing a decompensation event, as compared to patients identified with a lower risk of developing a decompensation event.

39. The method of claim 38, further comprising promoting the priority of the patient on a transplant list if the patient is identified as having high risk of developing a decompensation event vis-à-vis patients identified with a lower risk of developing a decompensation event.

40. The method of claim 38, wherein the predetermined threshold value is 5.5%/h.

41. The method of claim 38, wherein the decompensation event comprises occurrence of at least one of: new onset or worsening of hepatic encephalopathy, new onset or refractory ascites, variceal hemorrhage or occurrence of large gastroesophageal varices or any varices with red wale sign, portal hypertensive gastropathy hemorrhage, spontaneous bacterial peritonitis, hepatorenal syndrome, at least 2-point progression in MELD score, liver-specific or all-cause death or any combination thereof.

42. The method of claim 38, wherein the patient is suffering from cirrhotic NASH cirrhosis or from decompensated cirrhotic NASH.

43. The method of claim 38, wherein the method comprises identifying the patient as having a high-risk of experiencing a decompensation event within 6 months of the breath-test, if the PDR-peak value is at or below the predetermined threshold value.

44. The method of claim 38, further comprising providing a monthly accumulated risk score, based on the calculated PDR-peak value.
